(19) United States
(12) Patent Application Publication
(10) Pub. No.: US 2024/0350197 A1
(43) Pub. Date: Oct. 24, 2024

(54) METHOD AND APPARATUS FOR SAFE AND EFFICACIOUS TREATMENT OF UROLOGICAL CONDITIONS WITH LASER ENERGY

(71) Applicant: IPG PHOTONICS CORPORATION, Marlborough, MA (US)

(72) Inventors: Gregory ALTSHULER, Lincoln, MA (US); ILYA YAROSLAVSKY, N. Andover, MA (US); Viktoriya ANDREEVA, Fryazino (RU); Anastasia KOVALENKO, Wellesley, MA (US); Kevin COYLE, Arlington, MA (US); Olivier TRAXER, Paris (FR); Alexander VYBORNOV, Wellesley, MA (US)

(73) Assignee: IPG PHOTONICS CORPORATION, Marlborough, MA (US)

(21) Appl. No.: 18/683,700

(22) PCT Filed: Aug. 24, 2022
(86) PCT No.: PCT/US22/41375

§ 371 Date: Feb. 14, 2024

Related U.S. Application Data
(60) Provisional application No. 63/236,514, filed on Aug. 24, 2021.

(51) Int. Cl. A61B 18/26; A61B 18/00

(52) U.S. Cl. CPC A61B 18/26; A61B 2018/00511; A61B 2018/0066; A61B 2018/00702; A61B 2018/00898

(57) ABSTRACT

A laser system that includes a processing laser configured to generate a laser beam, a beam delivery system configured to direct the laser beam at a target, a user input device configured to receive input from a user, and a controller coupled to the processing laser and the user input device and configured to: receive initial user input data from the user input device, the initial user input data including at least one of: one or more properties of the beam delivery system, and one or more properties of the target, determine at least one initial laser operating parameter value and a corresponding initial laser operating parameter range based on the initial user input data, and electronically stored information, and control the processing laser using the at least one initial laser operating parameter value.

### RELATED APPLICATIONS

The present application claims priority to U.S. Provisional Application Ser. No. 63/236,514, titled “METHOD AND APPARATUS FOR SAFE AND EFFICACIOUS TREATMENT OF UROLOGICAL CONDITIONS WITH LASER ENERGY,” filed on Aug. 24, 2021 the content of which is herein incorporated by reference in its entirety.

### BACKGROUND

### Technical Field

The technical field relates generally to the treatment of urological conditions with directed laser energy.

### Background Discussion

Kidney stone disease is a prevalent condition estimated to be affecting 12% of the world population. Although most of the patients can pass the stones naturally, the condition can be severe enough that would require medical intervention. Extreme pain, nausea, vomiting, infection, blockage of urine flow and loss of kidney function can follow. Laser lithotripsy is a method for treatment of kidney stones. Laser energy directed through an optical fiber is used to break the stone into finer parts that can be passed naturally.

One of the major problems of laser lithotripsy includes using enough laser energy to ablate the stone material but not also accidentally damage intact surrounding soft tissue. Laser energy is also widely used to treat various pathologies of soft tissues (e.g., benign prostate hyperplasia (BPH), bladder tumors, etc.) through ablation, incision, excision, vaporization, and coagulation of tissue. As in the case as described above with stones, optimal selection of the treatment parameters is a compromise between efficacy and safety. There remains a need for the ability to address these problems.

### SUMMARY

Aspects and embodiments are directed to a method and system for providing optimal laser parameters for treatment of urological conditions.

In accordance with an exemplary embodiment, there is provided a laser system that includes a processing laser configured to generate a laser beam, a beam delivery system configured to direct the laser beam at a target, a user input device configured to receive input from a user, and a controller coupled to the processing laser and the user input device and configured to: receive initial user input data from the user input device, the initial user input data including at least one of: one or more properties of the beam delivery system, and one or more properties of the target, determine at least one initial laser operating parameter value and a corresponding initial laser operating parameter range based on the initial user input data, and electronically stored information, and control the processing laser using the at least one initial laser operating parameter value.

In one example, the controller is configured to display the at least one initial operating parameter value and the corresponding initial laser operating parameter range on a display device.

In one example, the controller is further configured to: receive one or more adjusted laser operating parameter values from the user input device, determine at least one modified laser operating parameter value and corresponding modified laser operating parameter range based on the one or more adjusted laser operating parameter values, and the electronically stored information, and control the processing laser using at least one of the adjusted laser operating parameter value and the at least one modified laser operating parameter value.

In one example, the at least one laser operating parameter that is modified is different than the one or more laser operating parameters that is adjusted.

In one example, the initial laser operating parameter range and the modified laser operating parameter range are determined such that a lower limit value of each range corresponds to a minimum efficacy for the laser operating parameter, and an upper limit value of each range corresponds to a safety limit for the laser operating parameter. In a further example, the controller is configured to display the lower and upper limit values of each range on a display device.

In one example, the controller is further configured to display a system laser operating parameter range for each displayed laser operating parameter range. In a further example, the laser operating parameter range is narrower than the system laser operating range.

In one example, the controller is further configured to determine if the one or more adjusted laser operating parameter values received from the user input device exceed the upper limit value of either range, and in response to the one or more adjusted laser operating parameter values exceeding the upper limit of either range, perform at least one of: display a visual alarm on a display device, and sound an auditory alarm on an auditory device.

In one example, the controller is configured to determine the at least one modified laser operating parameter value and corresponding modified laser operating parameter range such that at least one of an initial laser operating parameter value, and a corresponding initial laser operating parameter range for one of the at least one initial laser operating parameters is held constant.

In one example, the one of the at least one initial laser operating parameters that is held constant is average power. In a further example, at least one of a minimum efficacy value and a safety limit value for the average power are held constant.

In one example, the one or more adjusted laser operating parameter values comprises at least one of: average power, pulse shape, pulse repetition rate, pulse energy, pulse duration, and peak power.

In one example, the target is a kidney stone and the one or more properties of the kidney stone comprise at least one of: stone location, stone procedure type, stone composition, stone hardness, and stone size. In a further example, the one or more properties of the beam delivery system comprises at least one of: surgical fiber core size, delivery system geometry, where the delivery system geometry is one of a rigid scope, a flexible scope, and a semi-rigid scope, distance between a fiber and the stone target, irrigation flow rate, and speed of fiber or laser beam movement relative to the stone target.

In one example, the controller is further configured to determine an effective average laser power (EAP) as a laser operating parameter according to the following expression: EAP=(AP*LOT)/LTT, where AP=average power, LOT=laser on time duration during LTT, and LTT=later treatment time, and display the EAP on a display device.

In one example, the controller is further configured to determine an effective duty cycle (EDC) as a laser operating parameter according to the following expression: EDC=LOT/LTT, where LOT=laser on time duration during LTT, and LTT=laser treatment time, and display the EDC on a display device.

In one example, the LTT is one of a time duration of an entire treatment procedure or a time duration of a portion of the treatment procedure.

In one example, the one or more properties of the beam delivery system comprises at least one of: surgical fiber core size, delivery system geometry, where the delivery system geometry is one of a rigid scope, a flexible scope, and a semi-rigid scope, distance between a fiber and the target, irrigation flow rate, and speed of fiber or laser beam movement relative to the target.

In one example, the one or more properties of the target comprises at least one of: target size, target location, target type, and target material.

In accordance with another exemplary embodiment, there is provided a method for controlling a processing laser, the processing laser configured to generate a laser beam and the method including receiving initial user input data, the initial user input data including at least one of one or more properties of a beam delivery system, the beam delivery system configured to direct the laser beam at a target, and one or more properties of the target, determining at least one initial laser operating parameter value and a corresponding initial laser operating parameter range based on the initial user input data, and electronically stored information, and controlling the processing laser using the at least one initial laser operating parameter value.

In one example, the method further includes displaying the at least one initial operating parameter value and the corresponding initial laser operating parameter range on a display device.

In one example, the method further includes receiving one or more adjusted laser operating parameter values, determining at least one modified laser operating parameter value and corresponding modified laser operating parameter range based on the one or more adjusted laser operating parameter values, and the electronically stored information, and controlling the processing laser using at least one of the adjusted laser operating parameter value and the at least one modified laser operating parameter value.

In one example, the at least one laser operating parameter that is modified is different than the one or more laser operating parameters that is adjusted.

In one example, the initial laser operating parameter range and the modified laser operating parameter range are determined such that a lower limit value of each range corresponds to a minimum efficacy for the laser operating parameter, and an upper limit value of each range corresponds to a safety limit. In a further example, the method includes displaying the lower and upper limit values of each range on a display device.

In one example, the method further includes determining if the one or more adjusted laser operating parameter values exceeds the upper limit value of either range, and in response to the one or more adjusted laser operating parameter values exceeding the upper limit value of either range, performing at least one of: displaying a visual alarm on a display device, and sounding an auditory alarm on an auditory device.

In one example, the at least one modified laser operating parameter value and corresponding modified laser operating parameter range are determined such that at least one of an initial laser operating parameter value, and a corresponding initial laser operating parameter range for one of the at least one initial laser operating parameters is held constant. In one example, the initial laser operating parameter range for average power is held constant. In one example, at least one of the minimum efficacy value and the safety limit value for the average power are held constant.

In one example, the one or more adjusted laser operating parameter values comprises at least one of: average power, pulse shape, pulse repetition rate, pulse energy, pulse duration, and peak power.

In one example, the target is a kidney stone and the one or more properties of the kidney stone comprise at least one of: stone location, stone procedure type, stone composition, stone hardness, and stone size.

In one example, the one or more properties of the beam delivery system comprises at least one of: surgical fiber core size, delivery system geometry, where the delivery system geometry is one of a rigid scope, a flexible scope, and a semi-rigid scope, distance between a fiber and the stone target, irrigation flow rate, and speed of fiber or laser beam movement relative to the stone target.

In one example, the method further includes determining an effective average laser power (EAP) as a laser operating parameter according to the following expression: EAP=(AP*LOT)/LTT, where AP=average power, LOT=laser on time duration during LTT, and LTT=later treatment time, and displaying the EAP on a display device.

In one example, the method further includes determining an effective duty cycle (EDC) as a laser operating parameter according to the following expression: EDC=LOT/LTT, where LOT=laser on time duration during LTT, and LTT=laser treatment time, and display the EDC on a display device.

Still other aspects, embodiments, and advantages of these example aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Embodiments disclosed herein may be combined with other embodiments, and references to “an embodiment,” “an example,” “some embodiments,” “some examples,” “an alternate embodiment,” “various embodiments,” “one embodiment,” “at least one embodiment,” “this and other embodiments,” “certain embodiments,” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 is a block diagram of a laser system in accordance with aspects of the invention;

FIG. 2A is one example of a set of initial laser operating parameters and corresponding ranges as calculated and displayed to a user in accordance with one or more aspects of the invention;

FIG. 2B is one example of a modified set of initial laser operating parameters and corresponding ranges as calculated and displayed to a user in accordance with one or more aspects of the invention;

FIG. 3 is a graph showing an efficacy/safety corridor for laser operating parameters in accordance with one or more aspects of the invention;

FIGS. 4A-4C are tables of optimal ranges and presets for kidney treatment using a Thulium fiber laser (TFL)-based system in accordance with aspects of the invention;

FIGS. SA and SB are tables of optimal ranges and presets for ureter treatment using a TFL-based system in accordance with aspects of the invention;

FIG. 6 is a table of optimal ranges and presets for bladder treatment using a TEL-based system in accordance with aspects of the invention;

FIG. 7 is a table of optimal ranges and presets for soft tissue treatment using a TFL-based system in accordance with aspects of the invention;

FIG. 8 is a table of the maximum laser settings allowed in accordance with aspects of the invention;

FIG. 9 is one example of a screenshot illustrating a graphical user interface that displays several laser operating parameters in accordance with aspects of the invention;

FIG. 10 is another example of a screenshot illustrating a graphical user interface that displays several laser operating parameters, including an effective average power, in accordance with aspects of the invention;

FIG. 11 is another example of a screenshot illustrating a graphical user interface that displays several laser operating parameters, including effective duty cycle, in accordance with aspects of the invention;

FIG. 12 is another example of a screenshot illustrating a graphical user interface that displays several laser operating parameters, including a minimum and maximum value for the average power;

FIG. 13 is one example of a screenshot illustrating a graphical user interface that displays examples of initial user input data in accordance with aspects of the invention; and

FIG. 14 is another example of a screenshot illustrating a graphical user interface that displays initial user input data, including characteristics of the beam delivery system, in accordance with aspects of the invention.

### DETAILED DESCRIPTION

Laser energy is widely used for the treatment of a variety of urological conditions, ranging between lithotripsy and soft-tissue surgeries (e.g., BPH removal). There remains a problem of selecting optimal (i.e., providing maximum efficacy yet also being safe) laser parameters for treatment. The systems and methods disclosed herein provide techniques for treating urological conditions using an evidence-or data-driven approach.

Experienced operators (surgeons) use their knowledge and expertise to set desired treatment parameters (e.g., laser operating parameters), but less experienced operators have to rely on preset values provided by the manufacturer of the laser system used. However, a single set of parameters cannot, by its very nature, constitute an optimum outcome for all realistic clinical conditions.

Disclosed herein are one or more embodiments of a laser system that comprises a laser source and a control unit providing the means to set optimal treatment parameters of the laser source. In one embodiment, specific optimal ranges and default set points for a Thulium Fiber Laser (TFL)-based system are provided. As described in further detail below, systems and methods in accordance with one or more embodiments provide the following:
Identify ranges of optimal laser operating parameters for a variety of clinical conditions;
Identify initial (default) treatment parameters within these ranges;
Program or otherwise provide these ranges and default parameter values into a graphical user interface (GUI) of a laser system;
Treat the targeted urological condition with the optimal parameters. The system is configured to allow the user to vary or otherwise adjust the laser parameters to adapt to the particular clinical situation on the one hand, but to ensure continuous safety and efficacy of the treatment on the other hand.

The embodiments described herein are directed to urology applications. However, it is to be appreciated that applications of these teachings to other medical fields is also within the scope of this disclosure.

### General System Description

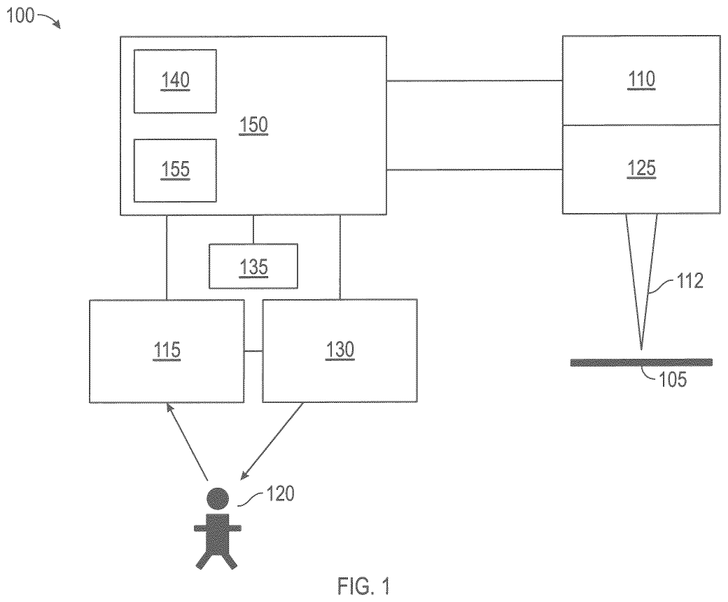

FIG. 1 is a block diagram of one non-limiting example of a laser system, shown generally at 100, that is provided by at least one embodiment. Laser system 100 comprises a processing (treatment) laser 110 configured to generate a laser beam 112, a beam delivery system 125 that is configured to direct the laser beam 112 at a target 105, a user input device 115 configured to receive input from a user 120, and a controller ISO coupled to the processing laser 110 and the user input device 115.

In accordance with at least one embodiment, laser energy from laser beam 112 and generated by the processing laser 110 is used to treat any one of a number of urological conditions, Non-limiting examples for the processing laser 110 include a Thulium-doped fiber laser (TEL), an Erbium-doped fiber laser, a Yttrium-doped fiber laser, a Ho:YAG solid-state laser, a Tm:YAG solid-state laser, or an Nd:YAG solid-state laser. Although not explicitly shown in FIG. 1, either controller 150 or the processing laser 110 itself comprises a driver for the laser source.

According to some embodiments, the beam delivery system 125 includes one or more components of a lithotripsy device, including a lithotripsy device configured to treat urological conditions. These components may comprise a delivery or surgical fiber that delivers the laser beam 112 to the target 105, and other accessories, including a delivery system (e.g., cystoscope, flexible endoscope with sheaths/no sheaths, (percutaneous nephrolithotomy (PCNL)/rigid endoscope, mini/ultramini PCNL endoscope), and other associated support components such as fluid flow devices (e.g., irrigation and aspiration functionality). In some embodiments, the beam delivery system 125 may comprise one or more optics, reflective devices, an articulated arm, and/or mechanical devices that are configured to direct the laser energy output from the processing laser 110 to the target 105.

The user input device 115 is configured to receive input from a user 120, such as a doctor and can take any one of a number of different forms, including a touchscreen. Besides a touch sensitive screen, other non-limiting examples of a user input device include a cursor control device (CCD), such as a mouse, a trackball, or joystick; a keyboard; one or more buttons, switches, or knobs; and a voice input system. According to at least one embodiment, input from the user constitutes user input data that can be used (at least in part) by controller 150 to control one or more components of the system 100, such as processing laser 110.

User input data may include initial user input data that is received from a user at the initiation of a procedure. In some embodiments, the initial user input data includes at least one of (1) one or more properties of the beam delivery system, and (2) one or more properties of the target.

In some embodiments, non-limiting examples of the one or more properties of the beam delivery system include a core size of the surgical fiber (e.g., surgical fiber core diameter), delivery system geometry (e.g., rigid scope or instrument, flexible scope or instrument, semi-rigid scope or instrument), an irrigation flow rate, a distance between a fiber and the target, and a speed of fiber or laser beam movement relative to the target.

According to some embodiments, non-limiting examples of the one or more properties of the target include target size, target location (e.g., for lithotripsy, the kidney, ureter, or bladder), target type, target procedure type, and target material. For instance, non-limiting examples of a target procedure type for kidney stone treatment include dusting, fragmentation, and popcoming, and for soft tissue treatment non-limiting example of the target procedure type include incision, excision, cutting, ablation, vaporization, prostate enucleation, and hemostasis. Non-limiting examples of target material include kidney stones and soft tissue. Other non-limiting examples of the target properties include location, composition, size, and hardness or density in the Hounsfield scale in instances where the target type is kidney stone. According to one embodiment, the target 105 is a kidney stone and the one or more properties of the kidney stone comprise at least one of stone location, stone procedure type, stone composition, stone hardness, and stone size.

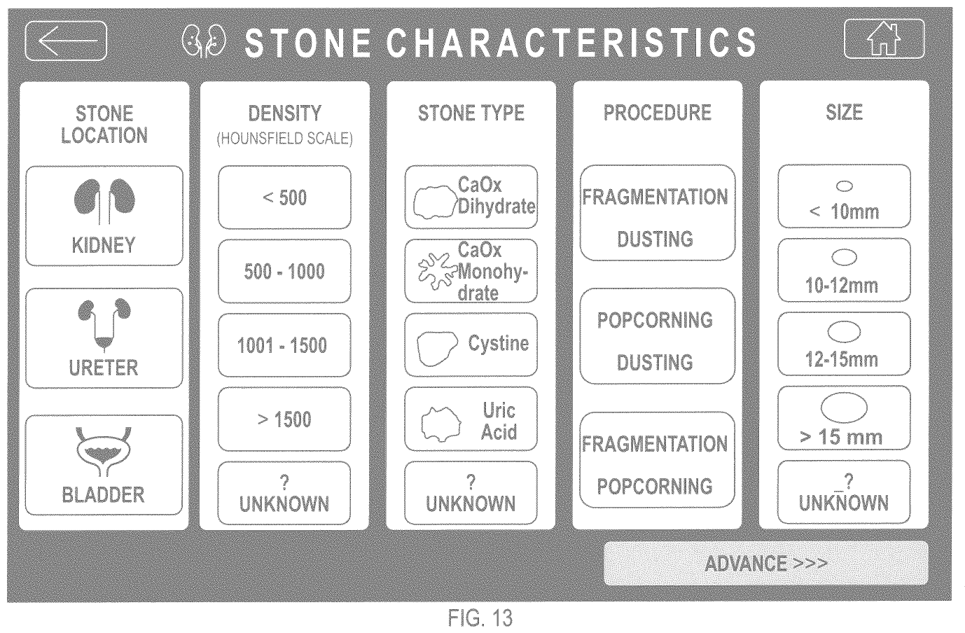

FIG. 13 is one example of a screenshot illustrating a graphical user interface that displays examples of initial user input data in accordance with one or more embodiments.

This initial user input forms at least a portion of an “Assistant Mode” (also referred to as “Assistance Mode”) offered to the user that helps the user perform a treatment operation in a safe yet effective manner. For example, in FIG. 13, and in accordance with at least one embodiment, the following options are listed for the user to use as initial user input data in a kidney stone treatment procedure for the stone target:

(A) stone location, e.g., kidney, ureter, or bladder,

(B) stone density in Hounsfield scale (also referred to herein as stone hardness), e <500, 500-1000, 1001-1500, >1500, or unknown,

(C) stone type (also referred to herein as atone composition), e.g., CaOx dihydrate, CaOx monohydrate, cystine, uric acid, or unknown

(D) stone procedure (procedure type) for both foot pedals, e.g., fragmentation and dusting, popcoming and dusting, or fragmentation and popcoming

(E) stone size, e.g., <10 mm. 10-12 mm, 12-15 mm, >15 mm, or unknown

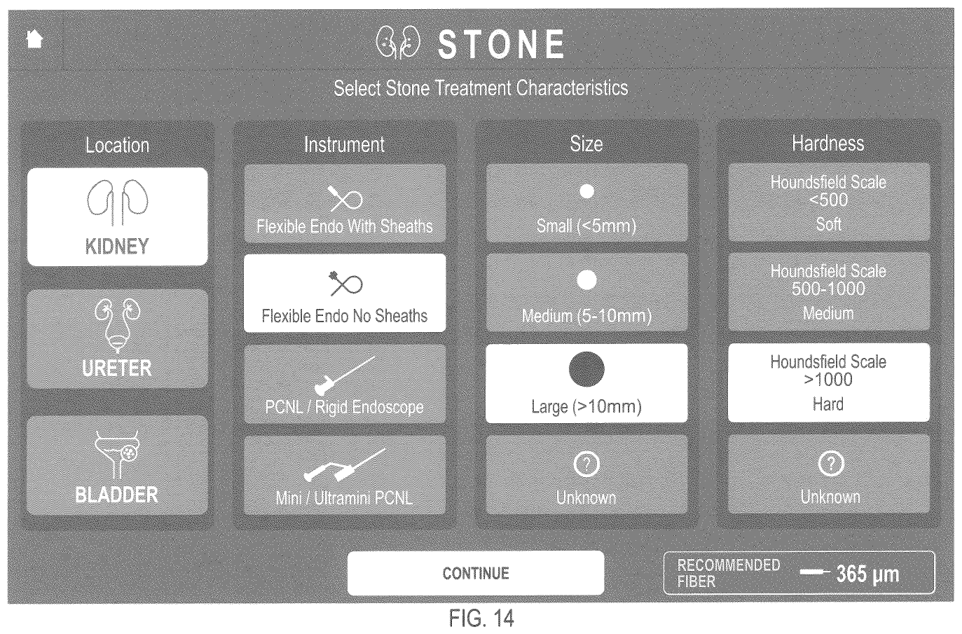

FIG. 14 is another example of a screenshot illustrating a graphical user interface that displays examples of initial user input data in accordance with one or more embodiments. For example, If a user selects “kidney” as the stone location in FIG. 13, then the screen in FIG. 14 is displayed, where the user can now select one or more properties regarding the beam delivery system 125. For example, in FIG. 14, and in accordance with at least one embodiment, the following options are listed to the user to use as initial user input data in a kidney stone treatment procedure for the beam delivery system:

(A) flexible Endo (endoscope) with Sheaths

(B) flexible Endo No Sheaths (which the user has selected and denoted by the lighter background color in this example shown in FIG. 14)

(C) PCNL/Rigid Endoscope

(D) Mini/Ultramini PCNL

Also included in FIG. 14 are target properties for the kidney stone, with the following non-limiting examples listed as potential options for the user in accordance with one embodiment:
Stone size, e.g., small (<5 mm), medium (5-10 mm), large (>10 mm), or unknown
Hardness (Hounsfield scale), e.g., soft (<500), medium (500-1000), hard (>1000), or unknown
   In this particular example shown in FIG. 14, the user has selected the large size and a hard stone.

10 The controller 150 is coupled to the processing laser 110 and the user input device 115. According to at least one embodiment, the controller 150 is configured to receive initial user input data from the user input device 115 and to determine at least one initial laser operating parameter value and a corresponding initial laser operating parameter range based on the initial user input data and electronically stored information. As discussed in further detail below, according to certain embodiments, the electronically stored information includes laser operating parameters for different target properties and beam delivery system configurations. In accordance with certain embodiments, non-limiting examples of laser operating parameters include average power, peak power, frequency (pulse repetition rate), pulse shape, pulse duration, and pulse energy.

The controller 150 includes circuitry that may be separate or integral components. It will be appreciated by those skilled in the art that the operations performed by the controller 150 may be performed by one or more controllers, processors, and/or other electronic components, including software and/or hardware components. For example, controller 150 includes a processor 155 (which may include more than one processor) and a computer-readable-storage device (not explicitly shown in FIG. 1), and a memory 140 (also referred to as a storage device), as well as other hardware and software components as will be appreciated by those of skill in the art.

As described above, the initial user input data can include one or more properties of the beam delivery system and/or target.

The electronically stored information is stored in a memory 140 of controller 150. In accordance with at least one embodiment, the electronically stored information may comprise look-up tables, empirical functions, and/or analytical models. This information is generated by inputting results from pre-clinical and clinical tests, trials and studies. In these embodiments, optimal ranges of laser operating parameters are based on objective evidence provided by pre-clinical and/or clinical studies. For example, an ex vivo (in vitro or phantom) environment mimicking the relevant clinical situation is created, and one or more of the laser operating parameters of interest (e.g., peak power, average power, pulse energy, repetition rate, pulse shape, fiber size, etc.) is varied to define the lower boundaries/limits of the corresponding laser operating parameter range which produce minimally acceptable stone ablation efficiency and rate (acceptable speed). Results are input as data into the memory 140 as stored information. According to at least one embodiment, the laser operating range has a lower limit or boundary that corresponds to a minimum efficacy for the laser operating parameter and an upper limit or boundary that corresponds to a safety limit or ceiling of the laser operating parameter. The safety limit is related to the acceptable risk of adverse effects, such as tissue damage through inadvertent action of the laser on tissue (e.g., organ wall perforation). Established ranges may be subsequently fine-tuned using actual clinical data with the corresponding laser type. This volume of data is then recorded as stored information in the system memory 140.

As an example, a variety of variables exist for different types of laser treatments. For lithotripsy, non-limiting examples of these variables include:
location of the stone (e.g., kidney, ureter, bladder)
material composition of the stone (e.g., calcium oxalate monohydrate (COM), urate etc.)
stone size
hardness of the stone (Hounsfield scale)
type of instrument available (rigid, flexible, etc.) dictating, in particular, the available range of irrigation flow rates

For soft-tissue surgery, non-limiting examples of these variables include:
location of the treatment site (e.g., kidney, ureter, bladder)
type of target (malignant, benign, etc.)
size of target
blood content of surrounding tissues
type of instrument available (rigid, flexible etc.)
procedure type such as hemostasis, incision, excision, ablation, vaporization, prostate gland enucleation, etc.

These variables are used in clinical studies to determine laser operating parameters that result in successful (and safe) treatment. One or more of these variables can be input as the initial user input data and used by controller 150 to access one or more corresponding electronically stored information that contains data generated by performing clinical studies (and has been input previously by a user and stored in memory 140).

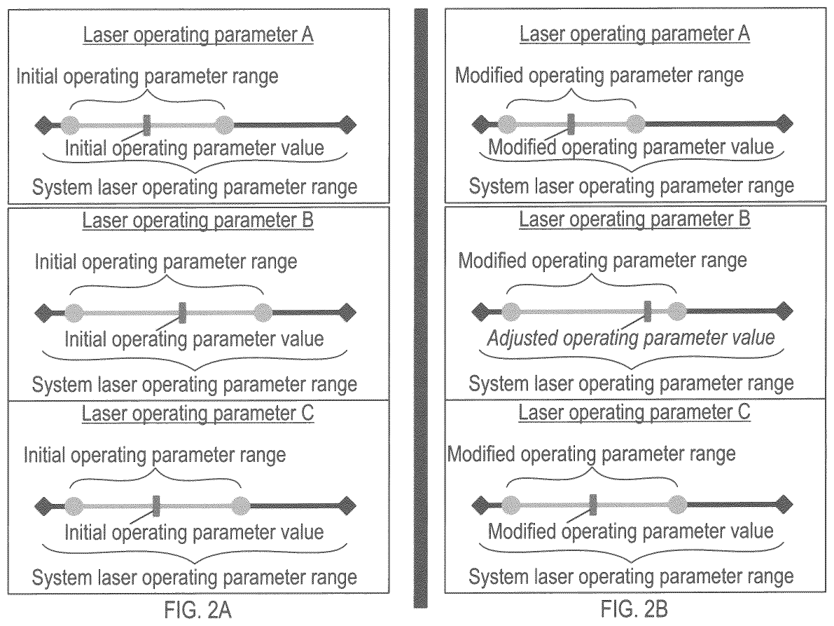

In some embodiments, once the controller 150 has determined the at least one initial laser operating parameter value and a corresponding initial laser operating parameter range, the controller 150 can display one or both of these parameters on a display device 130. The display device may provide three dimensional or two dimensional images and non-limiting examples include touch screen displays and/or flat panel displays or any other suitable visual output device capable of displaying graphical data and/or text to the user. In some embodiments a touch screen may function as both the display device 130 and as the user input device 115. According to at least one embodiment, the controller 150 is configured to generate on the display device 130 a graphical user interface (OUI) that receives user input in conjunction with the user input device 115. In accordance with at least one embodiment, FIG. 2A is one non-limiting example of a set of Initial laser operating parameters and corresponding ranges as calculated and displayed to a user 120 by the controller 150. This general layout may form at least a portion of a GUI used by the user 120 and the controller 150. In this example, the controller 150 determines three initial laser operating parameter values and their corresponding ranges (laser operating parameters A, B, and C). For each laser operating parameter, and in accordance with at least one embodiment. a system laser operating parameter range is also displayed. This “system” range is indicative of the system capabilities for that particular operating parameter and corresponds to a range typically displayed on conventional laser treatment systems. The minimum value in this “system” range is typically defined as the minimal laser system capability or minimum laser system parameter in this range (and not the minimum efficacy as disclosed herein). According to at least one embodiment, the initial operating parameter range is also displayed, which in some embodiments is narrower than the system operating range, and as discussed above, the lower limit value of this range corresponds to a minimum efficacy for the laser operating parameter and the upper limit value corresponds to a safety limit for the laser operating parameter. Also displayed is the initial operating parameter value. As discussed above, the initial operating parameter value and corresponding range are determined based on the initial user input data and electronically stored information. The initial operating parameter values and corresponding ranges, as well as the modified operating parameters and corresponding ranges described below form at least another portion of an “Assistant Mode” offered to the user that helps the user perform a treatment operation in a safe yet effective manner.

According to some embodiments, the default or initial laser operating parameter values may be defined in a variety of ways. For example, the merit figures of efficacy (E) and safety(S) can be assigned to each operating parameter. Typically, the E number will increase with increasing intensity of laser action, whereas the S number will correspondingly decrease. The intersection of the E and S curves provides an initial set point. Again, this set point may be fine-tuned using previous clinical experience.

The controller 150 is also configured to control the processing laser 110 using the at least one initial laser operating parameter value. Using FIG. 2A as an example, controller 150 would use the initial laser operating parameter values for operating parameters A, B, and C to control the processing laser 110. For instance, laser operating parameter A may be average power, laser operating parameter B may be pulse energy, and laser operating parameter C may be frequency. Both FIGS. 2A and 2B (described below) may form a portion of a GUI used by controller 150 and the user 120. The GUI is further characterized by providing a menu of treatment options and treatment locations (e.g., options for the initial user input data) to the user 120 at an initiation of a procedure (e.g., see FIGS. 13 and 14).

During a procedure, the user 120 may wish to change or otherwise adjust one or more of the laser operating parameter values, According to one embodiments, the one or more adjusted laser operating parameter values comprises at least one of average power, pulse shape, pulse repetition rate, pulse energy, pulse duration, and peak power. In accordance with one embodiment, controller 150 is configured to receive the one or more adjusted laser operating parameter values from the user input device 115. The controller 150 then 25 determines at least one modified laser operating parameter value and corresponding modified laser operating parameter range based on the one or more adjusted laser operating parameter values and electronically stored information. In some embodiments, the at least one laser operating parameter that is modified is different than the one or more laser operating parameters that is adjusted. FIG. 2B can be used to explain this functionality. FIG. 2B is one non-limiting example of the set of initial laser operating parameters A, B, and C (from FIG. 2A), where the user 120 has adjusted operating parameter value B, and controller 150 calculates or otherwise determines modified operating parameter values A and C and corresponding ranges that are displayed to the user 120. In some embodiments, controller 150 also determines a modified operating parameter range for the adjusted operating parameter value, which is also displayed to the user 120.

One important feature to appreciate is that in many embodiments laser operating parameters A, B, and C are interconnected with one another such that when one laser operating parameter is adjusted by the user, other laser parameters are modified in response to this change. One principal behind this interconnection is related to physics. For example, average laser power can be expressed by the expression:

$$Average Power ( A P ) = Frequency ( F ) * Total Pulse Energy ( T P E )$$

where
AP is in units of Watts (W),
F is in wits of Hertz (Hz), and
TPB is in units of Joules (J)

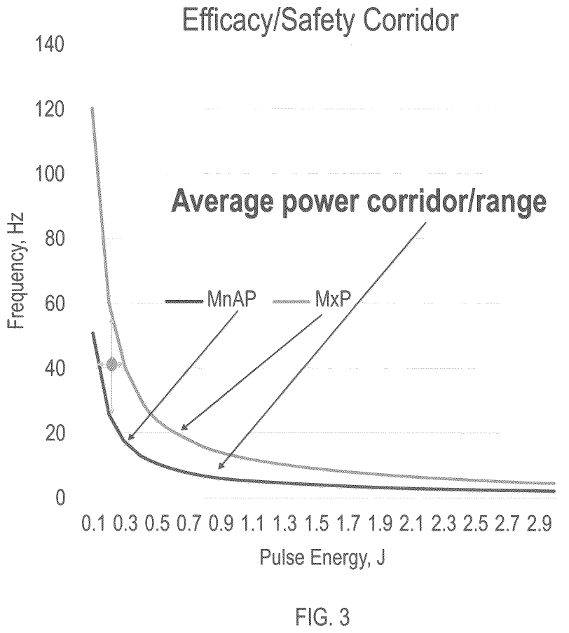

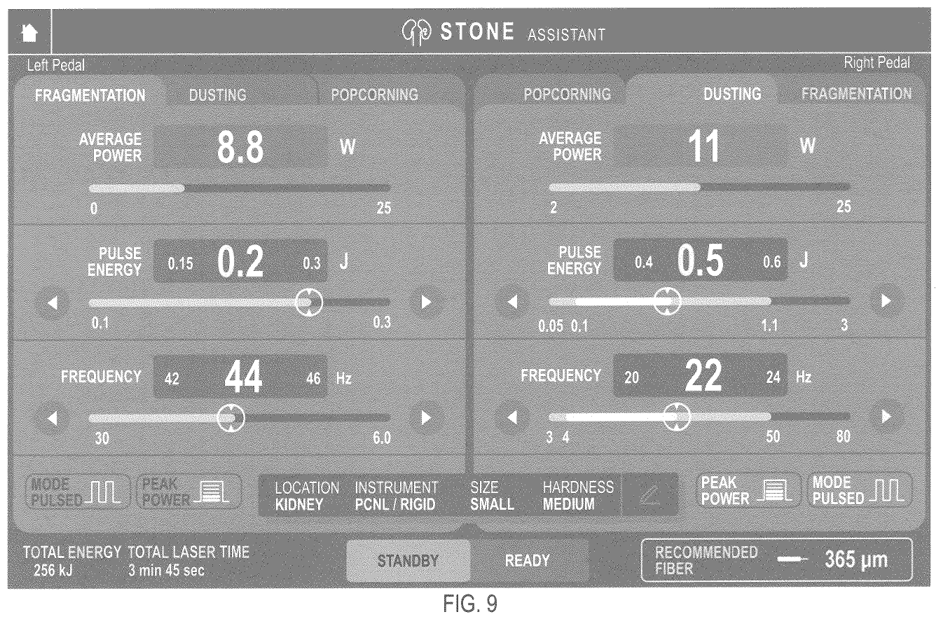

Another principal behind the interconnection of the laser operating parameters has to do with the concept of an efficacy/safety corridor that forms the basis for the laser operating parameter range (for both the initial and modified ranges as described above) and is implemented herein in certain embodiments. The efficacy/safety corridor is based on pre-clinical and/or clinical data as discussed above and is an example of information stored in the electronically stored information. FIG. 3 is a graph that helps explain this concept, where frequency (Hz) is labeled on the y-axis and pulse energy (J) is labeled on the x-axis. The curves indicate a maximum safe average laser power (M×P) and a minimum efficacious average laser power (MnAP) and the operating “corridor” for the average power is the space between these two lines, one example of which is shown in the double-arrowed line. For the example shown in FIG. 3 (which also corresponds to FIG. 9), the average power value is 8.8 W, which is a value that is in between the minimum efficacy value and the maximum safety limit value. In addition, the pulse energy is 0.2 J, and the frequency is 44 Hz. A corresponding range within the “efficacy/safety corridor” for the pulse energy is approximately 0.1-0.3 J, and a corresponding range for the frequency is approximately 25-55 Hz, where the lower limits correspond to the minimum efficacy and the upper limits correspond to the safety limit. Going back to FIG. 2B, when the user adjusts parameter B (e.g., frequency), the controller 150 modifies the other laser operating parameters A and C and their corresponding ranges such that all operating parameter values stay within the efficacy/safety corridor. This capability creates a dynamic response to a user's changes and ensures continuous safe and efficacious treatment.

The controller 150 is also configured to control the processing laser 110 using at least one of the adjusted laser operating parameter value and the at least one modified laser operating parameter value. Using FIG. 2B as an example, in some embodiments controller 150 would use the adjusted laser operating parameter B as well as the modified laser operating parameter values A and C to control the processing laser 110.

According to some embodiments, the controller 150 is also configured to determine if the one or more adjusted laser operating parameter values received from the user input device 115 exceeds the upper limit value of one or both of the initial laser operating parameter range and the modified laser operating parameter range, and in response to the one or more adjusted laser operating parameter values exceeding the upper limit value of either range, perform at least one of display a visual alarm on the display device 130, and sound an auditory alarm on an auditory device 135 (e.g., a speaker).

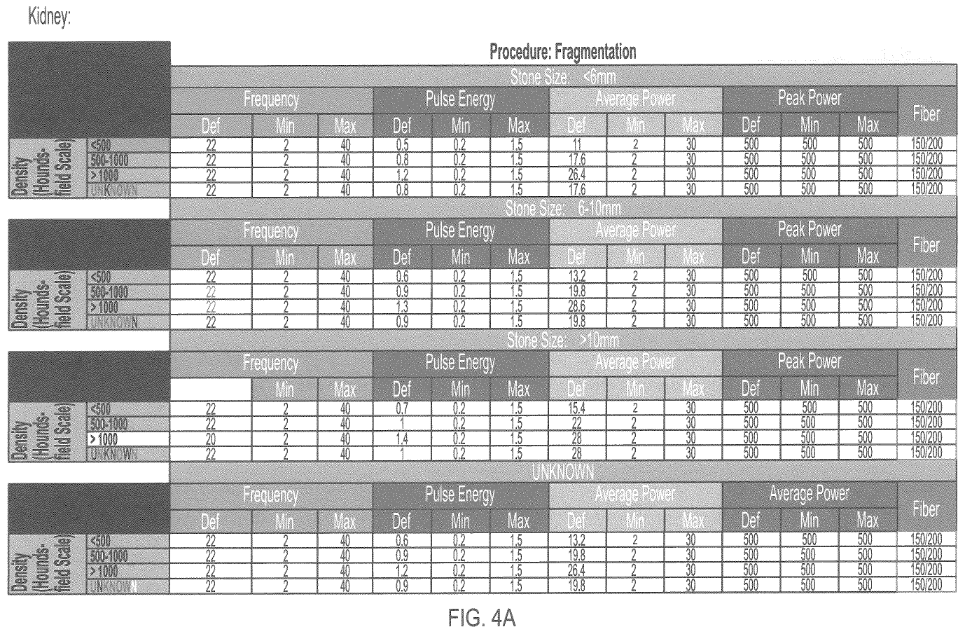

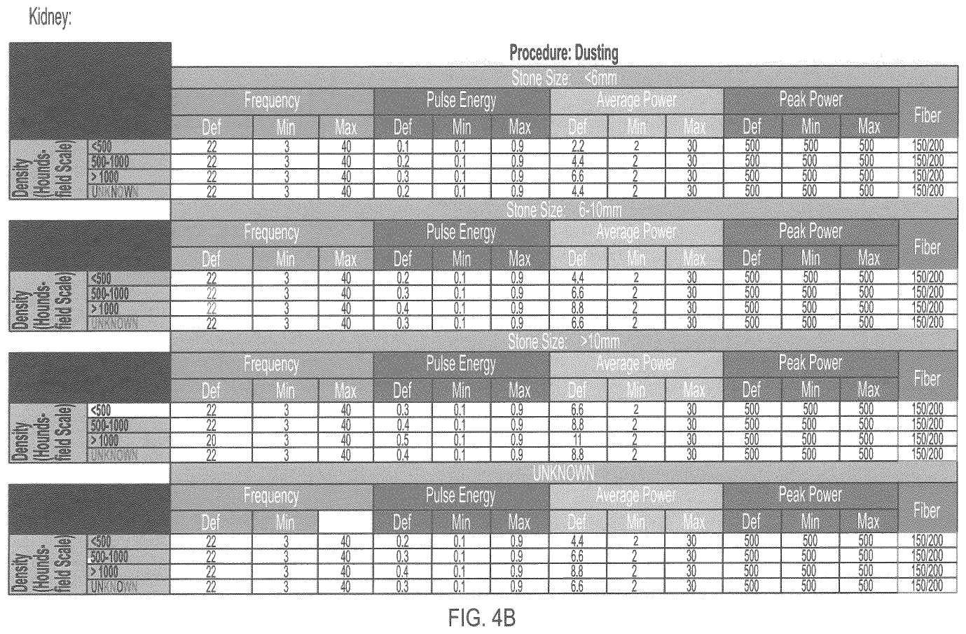

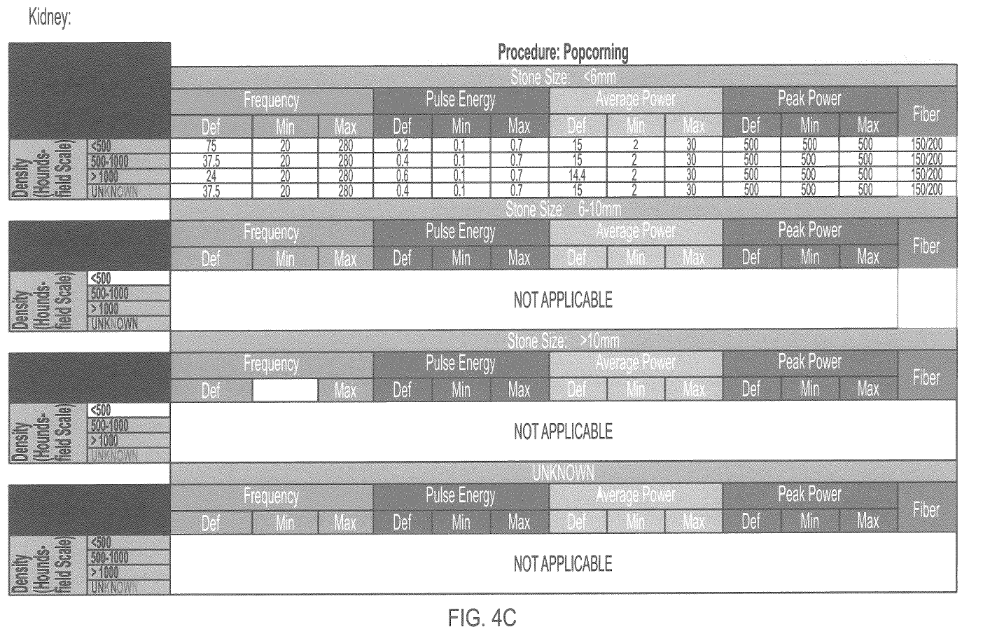

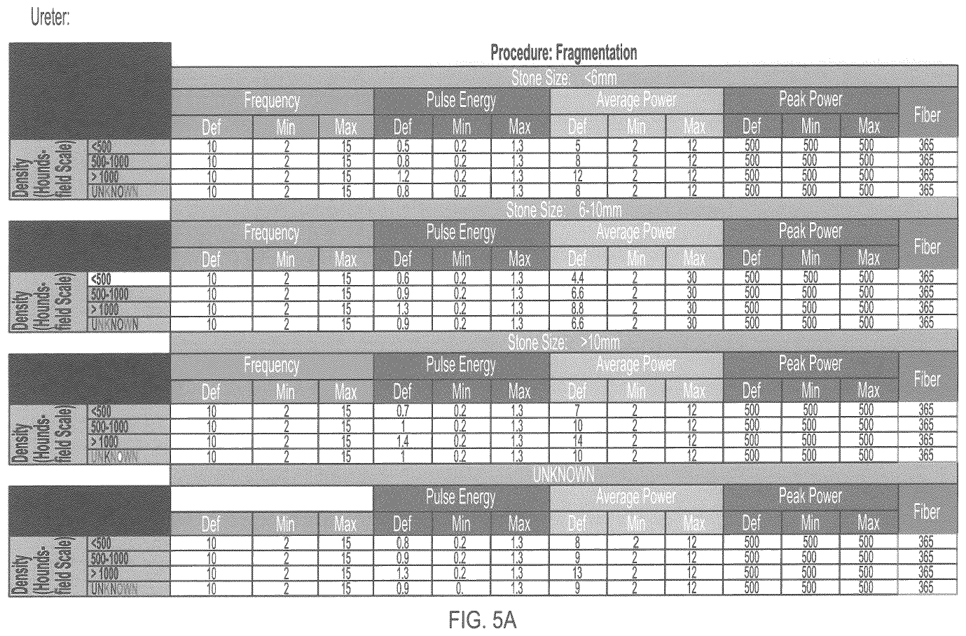

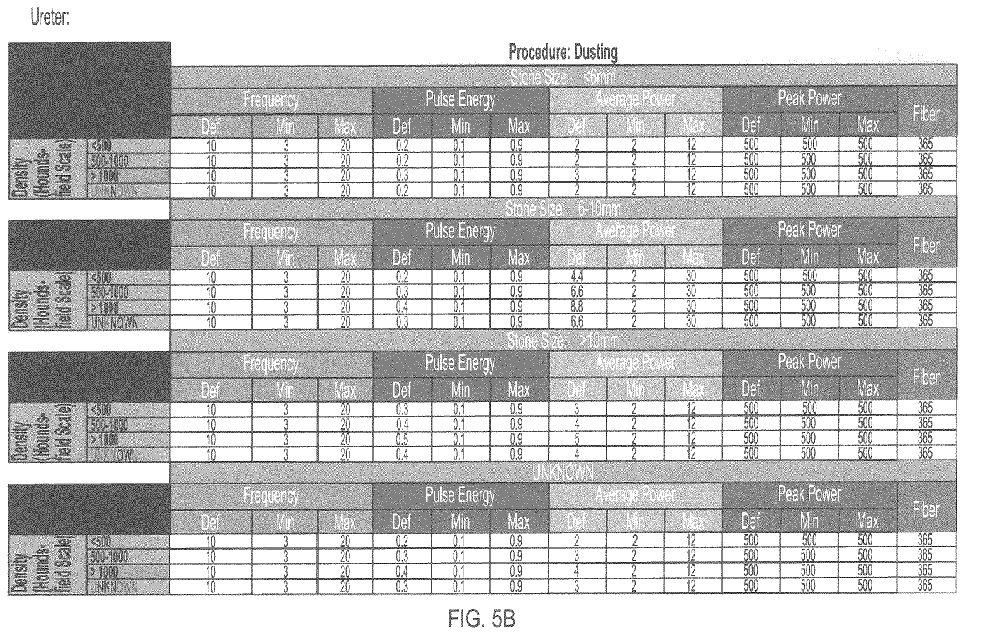

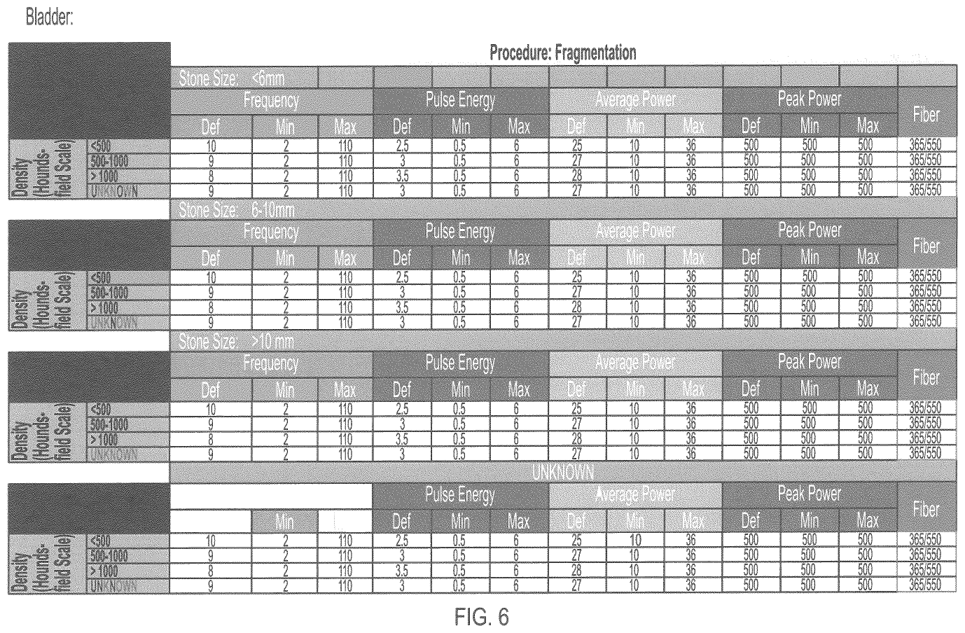

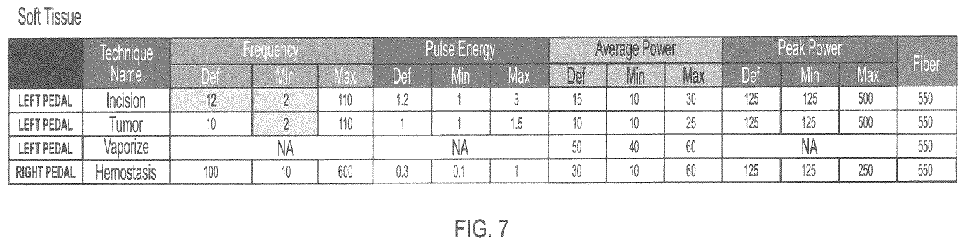

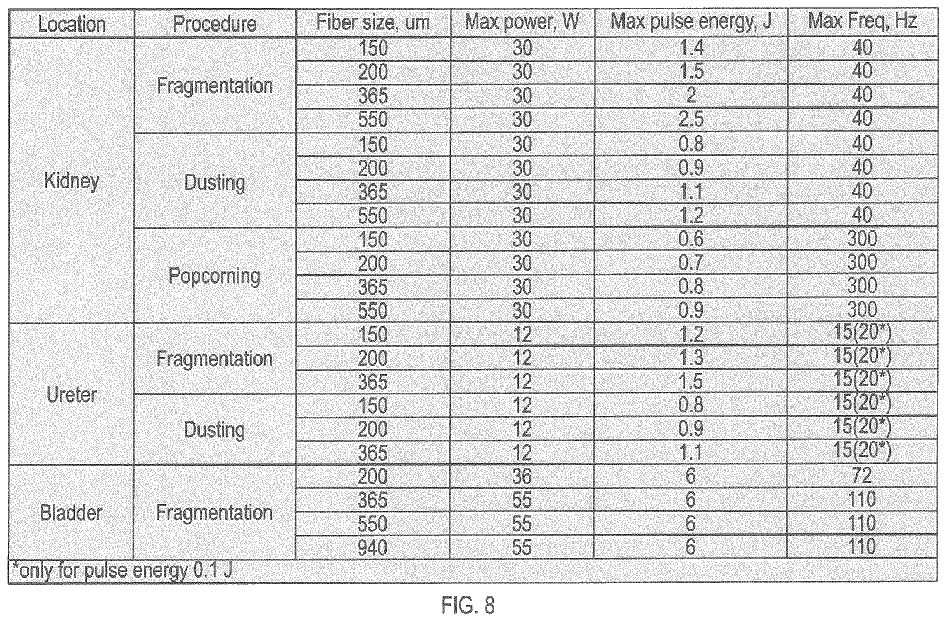

In accordance with certain embodiments, examples of TEL “Assistance Mode” laser operating parameter ranges are described herein. Experimental evaluation of the minimum TEL laser parameters required for minimal hard and soft tissue ablation efficiency was conducted using an in vitro model of stone (Bego stone). The maximum TFL operating parameters (defined per safety criteria as laser parameters required for perforation depths that exceed the thickness of the organ wall and for temperature increase of the ambient fluid in the organ increasing above 8°° C. of a normal body temperature) were conducted using an ex vivo kidney, ureter, and bladder volumetric model. The obtained ranges were verified by clinical studies and experiences of a panel of urologists. FIGS. 4A-4C, 5A, 5B, 6, and 7 show summarized sets of specific ranges and initial set points for a TEL-based treatment system for laser lithotripsy. In these tables, the minimum (Min), maximum (Max) (efficacy/safety corridor) and initial (Def) operating laser parameters are defined as frequency, pulse energy, average power, and peak power for different procedures, including fragmentation (FIGS. 4A, 5A, 6), dusting (FIGS. 4B, 5B), popcoming (FIG. 4C) in different locations corresponding to the kidney (FIGS. 4A-4C), ureter (FIOS. 5A, 5B), and bladder (FIG. 6). These laser parameters were correlated with the stone density in the Hounsfield scale and target size. FIG. 7 is one non-limiting example of summarized ranges of parameters and initial set points for soft tissue treatments, including tissue incision, tumor excision, tissue vaporization, and hemostasis. FIG. 8 is a table showing the maximum laser settings for the average power, pulse energy, and frequency for particular fiber sizes and treatment locations. All of the data shown in FIGS. 4-8 are examples of the type of data that comprises the electronically stored information as described herein.

During a procedure, the user 120 may wish to change or otherwise adjust the initial user input data. According to some embodiments, the adjusted initial user input data includes at least one of one or more adjusted properties of the beam delivery system 125 and one or more adjusted properties of the target 105. For instance, the user 120 may see a different type of stone material that needs to be treated with the processing laser 110, which would constitute an adjustment of a target property. In a similar manner as described above in reference to the adjusted laser operating parameter, controller 150 is configured to receive the adjusted initial user input data from the user input device 115 and determine at least one modified laser operating parameter value and corresponding modified laser operating parameter range based on the adjusted initial user input data and the electronically stored information. This process is analogous to the determination of the initial laser operating parameters described above and in the interest of brevity is not repeated here. In addition, the modified laser operating parameter value(s) and corresponding range(s) is displayed on the display device 130. According to some embodiments, once the modified values and ranges are displayed, the user 120 can either accept or reject these values. In this instance, the controller 150 is configured to receive user input (i.e., accept or reject) from the user input device 115 and then based on the user input, control the processing laser 110 using the at least one modified laser operating parameter. For instance, a user may accept or reject a modified laser power or pulse energy. If the user accepts the modified value, then the controller 150 controls the processing laser using this value. If the user rejects the modified value, then no change is made. In addition, if the resulting modified laser operating parameter value(s) exceeds the upper limit value corresponding to the safety limit, then a visual or auditory alarm is output by the controller 150.

Treatment Invariant within Dynamic GUI

In accordance with at least one embodiment, the dynamic response provided in the GUI (as previously described) is also configured with the concept of a treatment invariant. The treatment invariant is a laser operating parameter (or a set of laser operating parameters, or a range for a laser operating parameter) that is required to remain constant or in a defined range when the user adjusts one or more of the other laser operating parameters. In some embodiments, the controller 150 is configured to determine the at least one modified laser operating parameter value and corresponding modified laser operating parameter range such that at least one of an initial laser operating parameter value and a corresponding initial laser operating parameter range for one of the at least one initial laser operating parameters is held constant or in a defined range. In accordance with some embodiments, the initial laser operating parameter value and/or range that is held constant can be referred to as a treatment invariant.

Examples of treatment invariants include a maximum safe average laser power (M×P) and a minimum efficacious average laser power (MnAP). Besides individual values, the treatment invariant can include a range of values, which in this particular example includes a range for the average laser power where a minimum value is defined by MnAP, and a maximum value is defined by M×P. During a procedure, when the user attempts to change another laser operating parameter such as a pulse repetition rate (frequency) or a pulse energy, a modified or new average laser power is computed according to the formula as described previously (i.e., AP=F*TPE). If the resulting AP value is between the MnAP and the M×P (i.e., the treatment invariant range as described above), the change is allowed and the new laser operating parameters are sent to the laser by the controller. If, however, the resulting AP is outside of this range, then one or more of the laser operating parameters is modified to ensure that the treatment invariant range remains constant. If this is not possible, then the controller generates an alarm (e.g., visual and/or auditory alarm). Increasing the average laser power during treatment with a fixed water irrigation and output flow proportionally increases the temperature of water inside the organ (e.g., bladder, ureter, or kidney). If the temperature reaches above 45° C. and the exposure time increases beyond the safety limit the epithelium and connective tissue of the organ wall can coagulate, damaging the organ and causing severe side effects. Keeping the average power below the M×P value is considered to be very important to prevent such thermal damage during laser treatment.

FIG. 9 is an example of a screenshot of a GUI that helps explain this capability. In this example, the laser operating parameter related to the treatment invariant is the laser average power, which in this example has a treatment invariant range of 0-20 Watts (i.e., MnAP=2 and M×P=25, (not explicitly shown in FIG. 9). If the user attempts to adjust the pulse energy or the frequency and this results in the average laser power AP being outside of this treatment invariant range, then one or more other laser parameters are adjusted or otherwise modified to ensure that the average laser power stays within the treatment invariant range. For instance, in FIG. 9, a user may increase the pulse energy during a procedure that is inside the initial laser operating parameter range for the pulse energy (e.g., a range of 0.5 J to 3 J). Then the system automatically responds by changing the pulse frequency such that any selection within the frequency operating range allows for the laser average power to remain within the treatment invariant range (i.e., max of 25 W and 3 J=8.3 Hz in this example). This modified operating range for the frequency would then be displayed to the user. The user may increase the frequency during a procedure that is inside the initial laser operating parameter range (e.g., form 22 Hz to a maximum safe energy value of 80 Hz). Then the system automatically responds by changing the energy such that any selection within the energy operating range allows for the laser average power to remain within the treatment invariant range (e.g., 25 W/80 Hz=0.31 J in this example). This modified operating range for the energy would then be displayed to the user. If the one or more other laser parameters cannot be modified to ensure that the average power stays within the treatment range, then controller 150 is configured to perform at least one of display a visual alarm on the display device 130, and sound an auditory alarm on an auditory device 135.

### Other Laser Operating Parameters

Another example of a laser operating parameter that may be calculated by controller 150 and displayed to the user is the effective average laser power (EAP), which is computed according to the formula:

$$E A P = ( A P * Laser on Time ( L O T ) / Laser Treatment Time ( L T T )$$

where
EAP is in units of W,
LOT is in units of seconds(s) (during LTT, described below), and
LTT is in units of seconds.

In some embodiments the LTT is the time duration of an entire treatment procedure, and in other embodiments the LTT is the time duration of a portion of the treatment procedure.

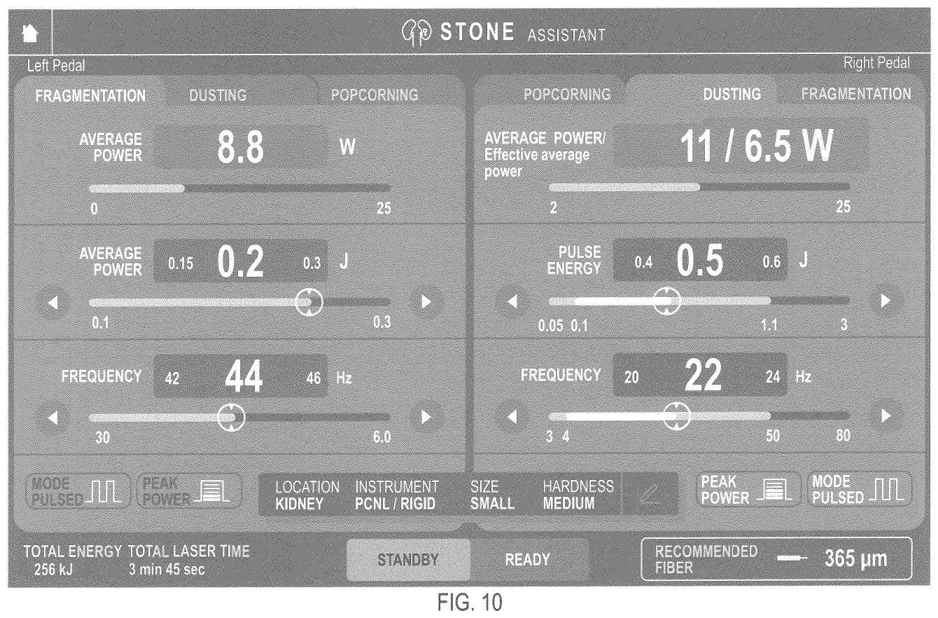

LOT is typically lower than LTT because during treatment the user is interrupting the laser firing using foot pedals at times when he or she needs to align the fiber within a desired distance to the target for purposes of preventing firing on surrounding tissue, or to allow the treatment area to be cleaned with irrigation flow for better visibility. EAP is a direct laser parameter which is used in determining the increase in temperature within the organ undergoing treatment. This temperature increase should be kept below a maximum safe level for a given treatment time. For example, if the average power of the laser system is set to be 30 W and the total treatment time between the first and last laser pulse delivered to the patient is 20 minutes, but the user switches on the laser for a total of 2, 10, and 15 minutes, the minimum EAP should be 6 W, 15 W, and 22 W, respectively. Since the temperature of the waste in an organ depends on the EAP, and not on AP, it is a significant benefit to provide the EAP to the user during a treatment procedure, FIG. 10 is a screenshot of one example of a GUI showing the display of both the average power and the EAP to the user on the right side of the screen.

Another example of a laser operating parameter that may be calculated by controller 150 and displayed to the user is the effective duty cycle (EDC), which is computed according to the formula:

$$E D C = L O T / L T T$$

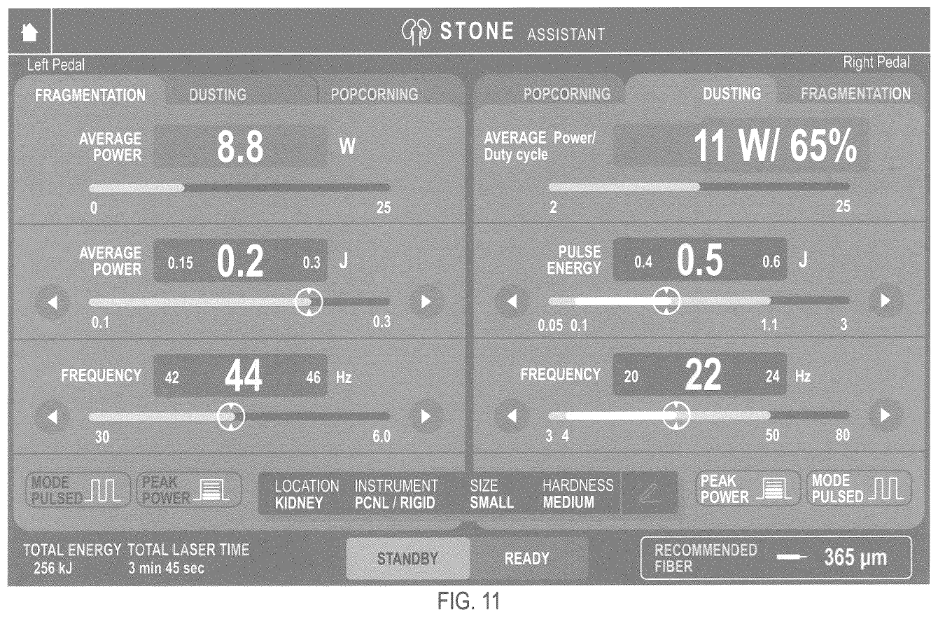

FIG. 11 is a screenshot of one example of a GUI showing the display of the average power and the EDC (also simply called “duty cycle”) to the user on the right side of the screen. The user can also estimate or calculate the EAP using the formula EAP=AP*EDC.

Both the EAP and the EDC are related to the actual amount of energy delivered to a patient's body during the treatment and are therefore included herein as relevant operating parameters. As with average power, invariant values and/or ranges for each of these parameters can also be calculated. In the case of the range, according to some embodiments the minimum value corresponds to minimum efficacy and the maximum value corresponds to a safety limit and one or both of these limits can be held constant. It is to be appreciated that the EAP and EDC are useful to consider only after a certain duration of time has passed in the treatment procedure (since both are dependent on the ITT). In this regard, and in accordance with certain embodiments, each of these laser operating parameters may be held constant at a time when the user performs an adjustment of another laser operating parameter (or adjusts the initial user input data), i.e., the controller 150 calculates or otherwise determines the EAP and/or EDC at the time the user makes their adjustment and holds EAP and/or EDC constant when determining the modified laser operating parameters and ranges. In a similar manner as described above, if an adjusted laser operating parameter (or any other adjusted input) results in an upper limit value for a range of either the EAP or EDC exceeding a safety limit, then controller 150 outputs an alarm (e.g., visual and/or auditory).

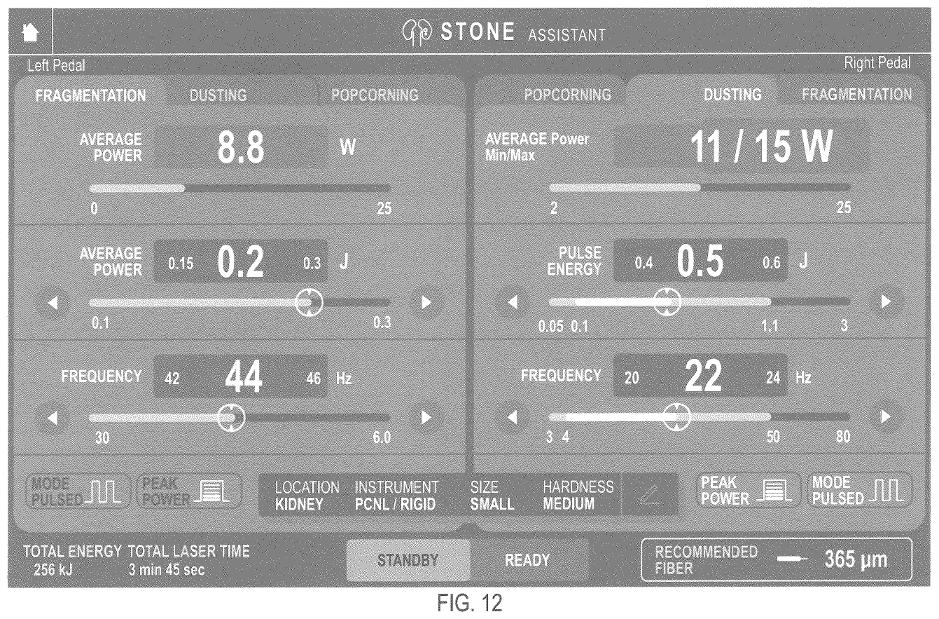

In accordance with another embodiment, another example of a laser operating parameter that may be displayed to the user includes minimum and maximum values for the average power. FIG. 12 is a screenshot of such a GUI displaying the Min and Max average power values to the user on the right side of the screen. The minimum and maximum values for the average power in this example correspond to the MAP and M×P curves shown in FIC. 3, respectively. In some embodiments, infographics of FIG. 3 (or its equivalent) can be directly displayed on the system screen (e.g., display device).

The aspects disclosed herein in accordance with the present invention, are not limited in their application to the details of construction and the arrangement of components set forth in the following description or illustrated in the accompanying drawings. These aspects are capable of assuming other embodiments and of being practiced or of being carried out in various ways. Examples of specific implementations are provided herein for illustrative purposes only and are not intended to be limiting. In particular, acts, components, elements, and features discussed in connection with any one or more embodiments are not intended to be excluded from a similar role in any other embodiments.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising.” “having.” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated reference is supplementary to that of this document; for irreconcilable inconsistencies, the term usage in this document controls. Moreover, titles or subtitles may be used in the specification for the convenience of a reader, which shall have no influence on the scope of the present invention.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

What is claimed is:

1. A laser system, comprising:
a processing laser configured to generate a laser beam;
a beam delivery system configured to direct the laser beam at a target;
a user input device configured to receive input from a user; and
a controller coupled to the processing laser and the user input device and configured to:
receive initial user input data from the user input device, the initial user input data including at least one of:
one or more properties of the beam delivery system, and
one or more properties of the target;
determine at least one initial laser operating parameter value and a corresponding initial laser operating parameter range based on
the initial user input data, and
electronically stored information; and
control the processing laser using the at least one initial laser operating parameter value.

2. The laser system of claim 1, wherein the controller is configured to display the at least one initial operating parameter value and the corresponding initial laser operating parameter range on a display device.

3. The laser system of claim 1, wherein the controller is further configured to:
receive one or more adjusted laser operating parameter values from the user input device;
determine at least one modified laser operating parameter value and corresponding modified laser operating parameter range based on
the one or more adjusted laser operating parameter values, and
the electronically stored information; and
control the processing laser using at least one of the adjusted laser operating parameter value and the at least one modified laser operating parameter value.

4. The laser system of claim 3, wherein the at least one laser operating parameter that is modified is different than the one or more laser operating parameters that is adjusted.

5. The laser system of claim 3, wherein the initial laser operating parameter range and the modified laser operating parameter range are determined such that
a lower limit value of each range corresponds to a minimum efficacy for the laser operating parameter, and
an upper limit value of each range corresponds to a safety limit for the laser operating parameter.

6. The laser system of claim 5, wherein the controller is configured to
display the lower and upper limit values of each range on a display device, and
display a system laser operating parameter range for each displayed laser operating parameter range

7. (canceled)

8. The laser system of claim 6, wherein the laser operating parameter range is narrower than the system laser operating range.

9. The laser system of claim S, wherein the controller is further configured to
determine if the one or more adjusted laser operating parameter values received from the user input device exceed the upper limit value of either range, and
in response to the one or more adjusted laser operating parameter values exceeding the upper limit of either range, perform at least one of:
display a visual alarm on a display device, and
sound an auditory alarm on an auditory device.

10. The laser system of claim 3, wherein the controller is configured to determine the at least one modified laser operating parameter value and corresponding modified laser operating parameter range such that
at least one of
an initial laser operating parameter value, and
a corresponding initial laser operating parameter range
for one of the at least one initial laser operating parameters is held constant.

11. The laser system of claim 10, wherein the one of the at least one initial laser operating parameters that is held constant is average power.

12. The laser system of claim 11, wherein at least one of a minimum efficacy value and a safety limit value for the average power are held constant.

13-15. (canceled)

16. The laser system of claim 3, wherein the controller is further configured to
determine an effective average laser power (EAP) as a laser operating parameter according to the following expression:
$$E A P = ( A P * L O T ) / L T T ,$$
where
AP=average power,
LOT=laser on time duration during LTT, and
LTT=later treatment time; and
display the EAP on a display device.

17. The laser system of claim 3, wherein the controller is further configured to
determine an effective duty cycle (EDC) as a laser operating parameter according to the following expression:
$$E D C = L O T / L T T ,$$
where
LOT=laser on time duration during LTT, and
LTT=laser treatment time; and
display the EDC on a display device.

18. (canceled)

19. The laser system of claim 1, wherein the one or more properties of the beam. delivery system comprises at least one of:
surgical fiber core size,
delivery system geometry, where the delivery system geometry is one of a rigid scope, a flexible scope, and a semi-rigid scope,
distance between a fiber and the target,
irrigation flow rate, and
speed of fiber or laser beam movement relative to the target.

20. The laser system of claim 1, wherein the one or more properties of the target comprises at least one of:
target size,
target location,
target type, and
target material.

21. A method for controlling a processing laser, the processing laser configured to generate a laser beam and the method comprising:
receiving initial user input data, the initial user input data including at least one of one or more properties of a beam delivery system, the beam delivery system
configured to direct the laser beam at a target, and
one or more properties of the target;
determining at least one initial laser operating parameter value and a corresponding initial laser operating parameter range based on
the initial user input data, and
electronically stored information; and
controlling the processing laser using the at least one initial laser operating parameter value.

22. The method of claim 21, further comprising displaying the at least one initial operating parameter value and the corresponding initial laser operating parameter range on a display device.

23. The method of claim 21, further comprising:
receiving one or more adjusted laser operating parameter values;
determining at least one modified laser operating parameter value and corresponding modified laser operating parameter range based on
the one or more adjusted laser operating parameter values, and
the electronically stored information; and
controlling the processing laser using at least one of the adjusted laser operating parameter value and the at least one modified laser operating parameter value.

24. The method of claim 23, wherein the at least one laser operating parameter that is modified is different than the one or more laser operating parameters that is adjusted.

25. The method of claim 23, wherein the initial laser operating parameter range and the modified laser operating parameter range are determined such that
a lower limit value of each range corresponds to a minimum efficacy for the laser operating parameter, and
an upper limit value of each range corresponds to a safety limit, and the method further comprises displaying the lower and upper limit values of each range on a display device

26. (canceled)

27. The method of claim 25, further comprising
determining if the one or more adjusted laser operating parameter values exceeds the upper limit value of either range; and
in response to the one or more adjusted laser operating parameter values exceeding the upper limit value of either range, performing at least one of:
displaying a visual alarm on a display device, and
sounding an auditory alarm on an auditory device.

28. The method of claim 23, wherein the at least one modified laser operating parameter value and corresponding modified laser operating parameter range are determined such that
at least one of
an initial laser operating parameter value, and
a corresponding initial laser operating parameter range
for one of the at least one initial laser operating parameters is held constant.

29. The method of claim 28, wherein the initial laser operating parameter range for average power is held constant.

30. The method of claim 29, wherein at least one of the minimum efficacy value and the safety limit value for the average power are held constant.

31-33. (canceled)

34. The method of claim 23, further comprising:
determining an effective average laser power (EAP) as a laser operating parameter according to the following expression:
$$E A P = ( A P * L O T ) / L T T ,$$
where
AP=average power,
LOT=laser on time duration during LTT, and
LTT=later treatment time; and
displaying the EAP on a display device.

35. The method of claim 23, further comprising:
determining an effective duty cycle (EDC) as a laser operating parameter according to the. following expression:
$$E D C = L O T / L T T ,$$
where
LOT=laser on time duration during LTT, and
LTT=Jas treatment time; and
display the EDC on a display device.

36. (canceled)
